(19) United States
(12) Patent Application Publication
(10) Pub. No.: US 2019/0150402 A1
(43) Pub. Date: May 23, 2019

(54) Dogs and Pets Diarrhea and Urine and Feces Cleaning Bags. Dogs and Pets' Urine Wipe Bags. Human Diarrhea and Feces Cleaning Bags. Dogs and Pets' Feces and Floor Cleaning Bags. Pet's Anus Cleaning Bags. Diarrhea, Feces and Urine Wipe Cleaning Bags

(71) Applicant: Albert Freeman Rockefeller, Chicago, IL (US)

(72) Inventor: Albert Freeman Rockefeller, Chicago, IL (US)

(21) Appl. No.: 15/815,738

(22) Filed: Nov. 17, 2017

(51) Int. Cl. A01K 23/00; B01J 20/24; A61L 15/42; A47L 13/16; A01K 13/00

(52) U.S. Cl. CPC A01K 23/005; B01J 20/24; A01K 13/001; A47L 13/16; A61L 15/42

(57) ABSTRACT

Bags with attachments inside of them to wipe and clean Diarrhea or feces or urine used for dogs, pets and humans to clean Diarrhea and feces or urine from the ground or from the body of elderly humans or infants or anyone in need that is unable to use the bathroom, also for hospital patients that can not use the bathroom. Also to clean anus and other dirty body parts. Bags are used to pickup feces and also to clean the floor with it as well or to clean urine from floor. It can be used to clean pets or animals' dirty eyes, nose, face paws or skin.

### DETAILED DESCRIPTION OF THE INVENTION

Dogs and Pets Diarrhea and Urine and Feces Cleaning Bags. Dogs and Pets' Urine wipe Bags. Human Diarrhea and Feces Cleaning Bags. Dogs and Pets' Feces and Floor Cleaning Bags. Pets' Anus cleaning Bags. Diarrhea, Feces and Urine wipe cleaning bags.

These bags with having an absorbent attachments into them are able to wipe clean diarrhea, urine and feces from ground or from anus area or hair of the pet or a from body of a human.

Current bags in use for pickup feces of pets does not have absorbent attachments so they are unable to pickup or absorb or wipe clean urine or diarrhea from the ground.

The absorbent attachment/s of bag help to wipe clean liquid from the ground or body.

It will wipe clean pets' urine and stops embarrassing the pet owners when their pet pees in a undesired areas like a shop or hallways of a building, elevators, etc.

cleaning of an elderly at home can be very challenging as may in the morning need to remove large amount of feces and diarrhea from body and the bags will help to wipe clean dirty areas and quickly close the bag and throw it away.

It can be used in kitchen or restaurants when there is a spill of a liquid or food.

Larger the animal is, larger bags can be used, as amount of diarrhea, urine or feces is different for different pets or animals. Larger bags with larger wiping absorbent attachments will be used for larger feces and bigger amount of diarrhea or urine in pets, animals or humans. Bag is made of lightweight material such as plastic and the attachment is made of absorbent material like cotton attached or glued to the other section. Sizes of the bags will vary for example normal dog diarrhea bags can be around 9×14 inches wide. Absorbent attachment for normal Anus Cleaning Bags can be around 2×2 wide inch, also absorbent Attachments can come in different shapes and sizes depend to what area specifically made for. Diarrhea and urine wipe cleaning bags have a larger absorbent section compare to just the Anus Cleaning Bags.

### BRIEF DESCRIPTION

Diarrhea and Feces and Urine wipe Cleaning bags will replace the current bags used with pet owners that are just useful to pickup the feces from ground but there are mostly always small part of feces left behind and the plastic bags are not able to wipe clean urine, Diarrhea and the whole feces from the ground. This invention help pets to stay cleaner as it can be used to wipe their anus or wipe urine from their body. Rats eat dog feces and my invention helps remove the feces leftover with current bags. It will help elderly's caretakers to wipe clean feces from elderly easy and then close the bag and dump it away. Even many elderly without helping others can wipe themselves easy as they be unable to go to bathroom.

### PURPOSE OF INVENTION

This bags will help pet owners to clean after their pets with being able to wipe diarrhea from ground or from the anus and hair around their anus.

Will help pet owners to wipe clean urine from embarrassing situations where for example a dog pees in the undesired area like in the hallway of the building etc.

It will help communities to stay cleaner as will remove dog feces from ground and won't let rats search for feces and eat the leftover feces from the ground.

It will help elderlies caretakers to wipe clean feces or diarrhea from the body of elderlies much easier and as soon as it wipes the feces, they can seal the bag and thrown away in garbage bag.

It can be used to wipe face of pets when dirty or their eyes. Then seal the bag and dump it.

### PROBLEM(S) THE INVENTION SOLVES

Help pet owners to wipe clean the ground when their pet is having diarrhea.

Help pet owners to wipe clean urine when their pet pees in an undesired area.

Help pet owners to wipe clean feces leftover after picking up the main parts of feces with plastic part and using the absorbing area of the bag to wipe clean from the ground so the feces leftover won't attract the rats or be left behind causing smell or walk over issues.

If feces won't get cleaned well, when we walk on them will attach into our shoes and when we walk home we will bring it home to our living areas.

Help caretakers to easily wipe clean the diarrhea or feces from body or elderlies and then just close the bag and throw it away. Won't even need to use gloves while wipe cleaning with my invention.

Wont need extra plastic bags while wipe cleaning diarrhea or feces from the body of the elderly or infants or anyone in need as you just need to wipe it and close the bag and throw it away.

### BACKGROUND OF THE INVENTION

Bags. Feces pickup bags. These bags are used to wipe clean Diarrhea, feces, urine from the ground or from the body or anus for pets, animals or humans.

### BRIEF DESCRIPTION OF DRAWING

FIG. 1. Is an example of bag and absorbent section. This is an example bag used for dogs to wipe clean faces from ground or anus of the dog.

FIG. 2. Is an example of bag and absorbent section. This is an example of bag used for wipe clean diarrhea or urine from the ground or diarrhea from the hair or body of the pet.

FIG. 3. Is an example of bag (2) and absorbent section (1). This is an example bag used for dogs to wipe clean faces from ground or anus of the dog.

FIG. 4. Is an example of bag (2) and absorbent section (1). This is an example of bag used for wipe clean diarrhea or urine from the ground or diarrhea from the hair or body of the pet.

FIG. 4. Is an example of bag (2) and absorbent section (1). This is an example of bag used for wipe clean diarrhea or urine from the ground or diarrhea from the hair or body of the pet.

FIG. 5. Is an example of bag (2) and absorbent section (1) from above. This is an example of bag used for wipe clean diarrhea or urine from the ground or diarrhea from the hair or body of the pet

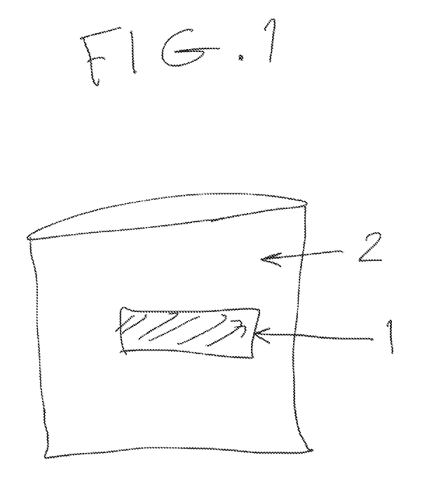

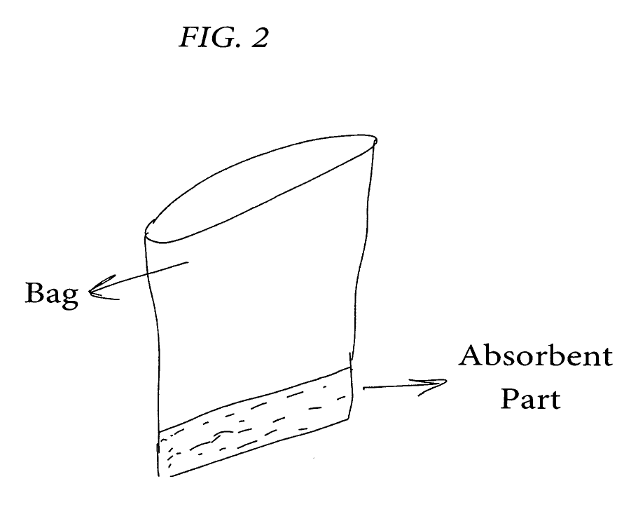

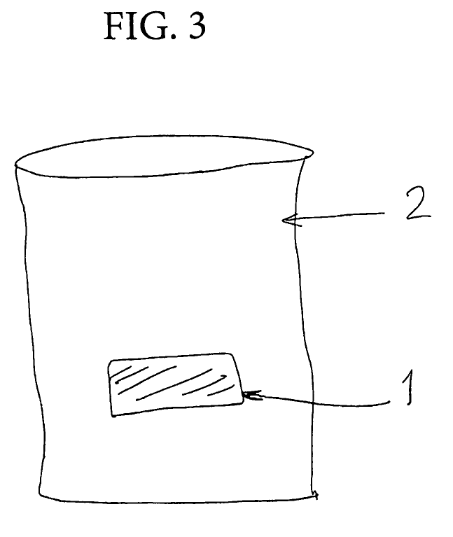

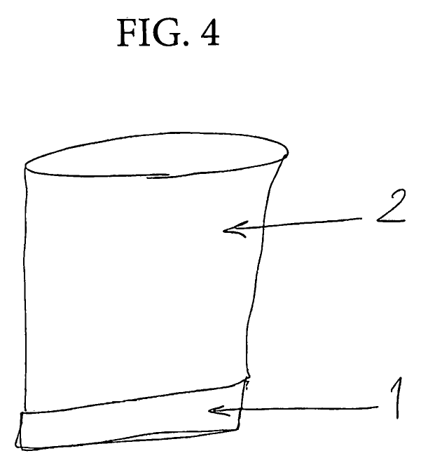

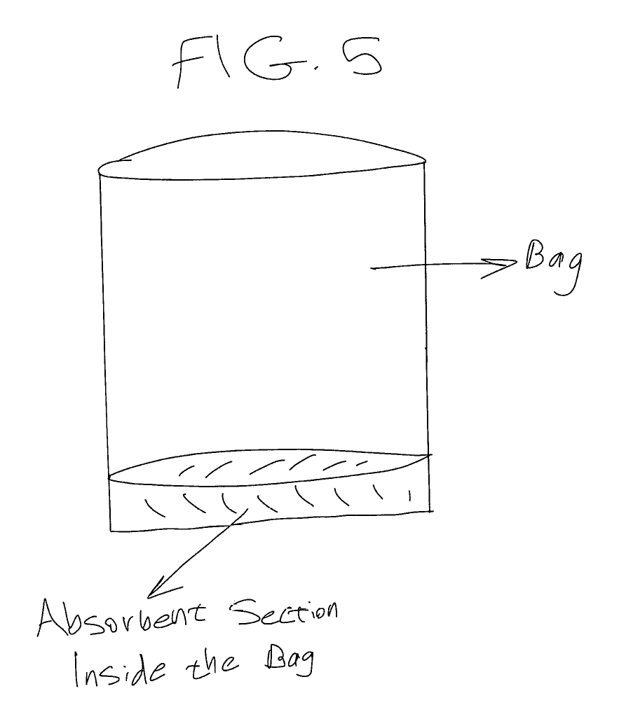

### TITLE OF INVENTION

Dogs and Pets Diarrhea and Urine and Feces Cleaning Bags. Dogs and Pets' Urine wipe Bags. Human Diarrhea and Feces Cleaning Bags. Dogs and Pets' Feces and Floor Cleaning Bags. Pets' Anus cleaning Bags. Diarrhea, Feces and Urine Wipe Cleaning Bags.

1. Dogs and Pets Diarrhea and Urine and Feces Cleaning Bags. Dogs and Pets' Urine wipe Bags. Human Diarrhea and Feces Cleaning Bags. Dogs and Pets' Feces and Floor Cleaning Bags. Pets' Anus cleaning Bags. Diarrhea, Feces and Urine wipe cleaning bags.
These bags with having an absorbent attachments into them are able to wipe clean diarrhea, urine and feces from ground or from anus area or hair of the pet or a from body of a human.
The absorbent attachment/s of bag help to wipe clean liquid from the ground or body.
It will wipe clean pets' urine and stops embarrassing the pet owners when their pet pees in a undesired areas like a shop or hallways of a building, elevators, etc.
cleaning of an elderly at home can be very challenging as may in the morning need to remove large amount of feces and diarrhea from body and the bags will help to wipe clean dirty areas and quickly close the bag and throw it away.
It can be used in kitchen or restaurants when there is a spill of a liquid or food. Larger the animal is, larger bags can be used, as amount of diarrhea, urine or feces is different for different pets or animals. Larger bags with larger wiping absorbent attachments will be used for larger feces and bigger amount of diarrhea or urine in pets, animals or humans. Bag is made of lightweight material such as plastic and the attachment is made of absorbent material like cotton attached or glued to the other section. Sizes of the bags will vary for example normal dog diarrhea bags can be around 9×14 inches wide. Absorbent attachment for normal Anus Cleaning Bags can be around 2×2 wide inch, also absorbent Attachments can come in different shapes and sizes depend to what area specifically made for. Diarrhea and urine wipe cleaning bags have a larger absorbent section compare to just the Anus Cleaning Bags.

2. A bag or bag-type container used for wipe clean or pickup diarrhea and urine from ground. A bag with absorbent attachments of different shapes and different types of absorbent material to wipe clean feces, Diarrhea and urine from ground or hair or body of pet, animal or human. A bag used for cleaning anus with using the absorbent attachment into or on it.

3. A bag according to claim 2, with different forms and sizes as the size depend on the amount of feces or diarrhea or urine needs to be cleaned. For example normal dog diarrhea bags can be around 9×14 inches wide. Absorbent attachment for normal Anus Cleaning Bags can be around 2×2 wide inch, also absorbent Attachments can come in different shapes and sizes depend to what area specifically made for. Diarrhea and urine wipe cleaning bags have a larger absorbent section compare to just the Anus Cleaning Bags.

4. A bag according to claims 1 and 2, with different shapes and sizes of absorbent attachments inside or outside the bag. Plastic or attachments can be from any color.
